(19) United States
(12) Patent Application Publication
(10) Pub. No.: US 2025/0318898 A1
(43) Pub. Date: Oct. 16, 2025

(54) RADIATION IMAGING APPARATUS AND MANUFACTURING METHOD

(71) Applicant: CANON KABUSHIKI KAISHA, Tokyo (JP)

(72) Inventors: TOMOHIRO HOSHINA, Kanagawa (JP); TAKAAKI GONDA, Kanagawa (JP); YOSHITAKA OTSUBO, Kanagawa (JP); AKIRA KIDA, Kanagawa (JP); YOSHITO SASAKI, Tokyo (JP)

(21) Appl. No.: 19/172,208

(22) Filed: Apr. 7, 2025

(30) Foreign Application Priority Data
Apr. 11, 2024 (JP) 2024-063718

(51) Int. Cl. A61B 90/00; A61B 6/42; G01T 7/00

(52) U.S. Cl. CPC A61B 90/08; A61B 6/4233; G01T 7/00

(57) ABSTRACT

A radiation imaging apparatus including a plurality of heat insulation members which are separate members including a first heat insulation member and a second heat insulation member, and a housing including an incident surface, a rear surface, and a side wall, wherein the first heat insulation member is disposed between a sensor panel and a board, wherein the second heat insulation member is disposed between a cable and the side wall, and wherein an internal space of the housing includes a space on a side with the incident surface and a space on a side with the rear surface, between which is a position where the first heat insulation member and the second heat insulation member hold the cable therebetween.

### BACKGROUND

### Field of the Disclosure

The present disclosure relates to a radiation imaging apparatus and a manufacturing method. The radiation imaging apparatus is used as, for example, a medical image diagnostic apparatus or an analysis apparatus.

### Description of the Related Art

Conventionally known radiation imaging apparatuses convert incident radiation into electric signals in a sensor panel and obtain a radiation image based on the electric signals.

Such a radiation imaging apparatus includes a large number of electrical components, and if heat generated from the electrical components is transmitted to the sensor panel, quality of a radiation image may be affected.

Japanese Patent No. 6778118 discusses a radiation imaging apparatus in which a heat insulation member is disposed between a sensor unit and an electrical component to reduce heat transmission from the electrical component to the sensor unit.

The conventional radiation imaging apparatus has room for improvement in terms of ease of assembly. This is because, in placement of the heat insulation member between the sensor unit and the electrical component, a flexible cable (wiring) connecting the sensor unit and the electrical component obstructs the placement of the heat insulation member. If the heat insulation member has a hole through which the flexible cable is passed, an operation of passing the flexible cable through the hole during assembly is cumbersome. Further, in a case of replacement of the flexible cable, it is troublesome to pull out the flexible cable from the heat insulation member.

### SUMMARY

Therefore, it is desirable that the radiation imaging apparatus in which the heat insulation member is disposed in the vicinity of the cable have a configuration excellent in ease of assembly.

Embodiments of the present disclosure are directed to providing a radiation imaging apparatus that has a heat insulation member in a vicinity of a cable and is still excellent in ease of assembly.

According to embodiments of the present disclosure, a radiation imaging apparatus includes a sensor panel configured to detect radiation, a cable configured to be connected to the sensor panel, a board configured to be connected to the sensor panel via the cable, a plurality of heat insulation members which are separate members including a first heat insulation member and a second heat insulation member, and a housing configured to house the sensor panel, the cable, the board, and the plurality of heat insulation members, the housing including an incident surface through which radiation is incident on the sensor panel, a rear surface on a side opposite to the incident surface, and a side wall connecting the incident surface and the rear surface, wherein the first heat insulation member is disposed between the sensor panel and the board, wherein the second heat insulation member is disposed between the cable and the side wall, and wherein an internal space of the housing includes a space on a side with the incident surface and a space on a side with the rear surface, between which is a position where the first heat insulation member and the second heat insulation member hold the cable therebetween.

Further features of the present disclosure will become apparent from the following description of exemplary embodiments with reference to the attached drawings.

### BRIEF DESCRIPTION OF THE DRAWINGS

FIG. 1 is a cross-sectional diagram illustrating an example of an internal configuration of a radiation imaging apparatus viewed from a side.

FIG. 2A is a diagram illustrating the radiation imaging apparatus viewed from a side on which radiation is incident, with an incident surface omitted. FIG. 2B is a diagram illustrating a cross section of the radiation imaging apparatus taken along a line A-A illustrated in FIG. 1.

FIG. 3 is a schematic diagram illustrating a housing during assembly.

FIG. 4 is a cross-sectional diagram illustrating another example of an internal configuration of a radiation imaging apparatus viewed from a side.

FIG. 5A is a diagram illustrating the radiation imaging apparatus viewed from a side on which radiation is incident, with an incident surface omitted. FIG. 5B is a diagram illustrating a cross section of the radiation imaging apparatus taken along a line A-A illustrated in FIG. 4.

FIG. 6 is a schematic diagram illustrating assembly of the housing.

FIG. 7 is a cross-sectional diagram illustrating yet another example of an internal configuration of a radiation imaging apparatus viewed from a side.

FIG. 8 is a cross-sectional diagram illustrating yet another example of an internal configuration of a radiation imaging apparatus viewed from a side.

FIG. 9 is a diagram illustrating a radiation imaging system.

### DESCRIPTION OF THE EMBODIMENTS

Hereinafter, exemplary embodiments of the present disclosure will be described in detail with reference to examples and drawings. The present disclosure is not limited to configurations described in the exemplary embodiments. Within a range in which the same effect is obtained, a part of the configuration or a part of the processing may be modified by being replaced with an equivalent or omitted.

### [Radiation Imaging System]

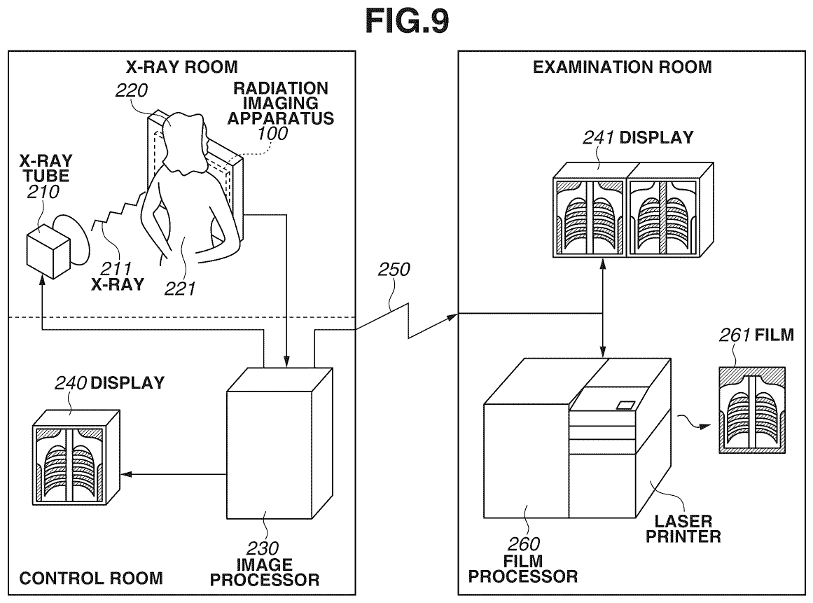

A first exemplary embodiment will be described. FIG. 9 is a diagram illustrating a configuration of a radiation imaging system. As illustrated in FIG. 9, radiation 211 (X-ray) generated by an X-ray tube 210 (radiation source, radiation irradiation apparatus) is transmitted through an imaging region 221 (chest) of a patient 220 (subject) and is incident on a radiation imaging apparatus 100 (radiation imaging apparatus, radiation detection apparatus). The incident X-ray include information on the inside of the body of the patient 220. A scintillator (phosphor) emits light in response to the incidence of the X-ray, and a sensor (photoelectric conversion element) of a sensor panel photoelectrically converts the light emission to obtain electrical information. The electrical information is converted into digital information, and the digital information is subjected to image processing by an image processor 230 (signal processing unit), whereby the digital information is observable via a display 240 (display unit). In this specification, radiation includes not only X-rays but also α-rays, β-rays, γ-rays, particle beams, and cosmic rays.

The information subjected to the image processing by the image processor 230 is transferred to a remote location by a transmission processing unit 250, such as a network including a telephone line, a local area network (LAN), and the Internet. Thus, information subjected to the image processing by the image processor 230 is displayed on a display unit 241 (display unit) in an examination room or the like in another place, or is stored in a recording unit, such as an optical disk, and is diagnosed by a doctor or others in a remote place. The information subjected to the image processing by the image processor 230 is also able to be recorded on a film 261 by a film processor 260.

### [Radiation Imaging Apparatus]

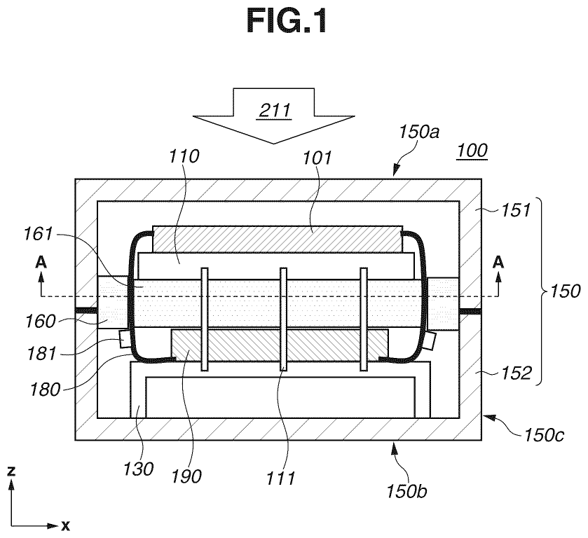

The radiation imaging apparatus 100 will be described. FIG. 1 is a sectional diagram illustrating an example of an internal configuration of the radiation imaging apparatus 100 viewed from a side.

The radiation imaging apparatus 100 (radiation imaging apparatus, radiation detection apparatus) is an apparatus for generating a radiation image based on radiation emitted from the X-ray tube 210 (radiation source, radiation irradiation apparatus). As illustrated in FIG. 9, the radiation imaging apparatus 100 in the present exemplary embodiment is a flat panel detector (FPD) having a flat plate shape. For convenience of illustrating the configuration of the radiation imaging apparatus 100 in an easy-to-understand manner, the length of the radiation imaging apparatus 100 in a thickness direction (z direction) is emphasized in each drawing including FIG. 1.

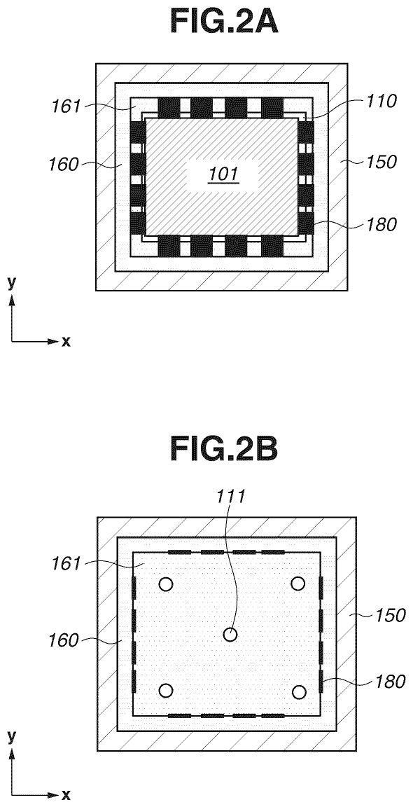

FIG. 2A is a diagram illustrating the radiation imaging apparatus 100 viewed from a side on which radiation is incident, with an incident surface omitted. FIG. 2B is a diagram illustrating a cross section of the radiation imaging apparatus 100 taken along a line A-A illustrated in FIG. 1.

The radiation imaging apparatus 100 includes a component illustrated in FIG. 1, and more specifically, includes a sensor panel 101, a base 110 (support base), support columns 111, a base 130, a housing 150, heat insulation members 160 and 161, cables 180, and a mounting board 190.

The housing 150 is a casing that houses an internal configuration (internal component) of the radiation imaging apparatus 100. The internal configuration includes the sensor panel 101, the base 110, the support columns 111, the base 130, the heat insulation members 160 and 161, the cables 180, the mounting board 190. The housing 150 has an incident surface 150a through which radiation is incident on the sensor panel 101, a rear surface 150b on the opposite side to the incident surface 150a, and a side wall 150c connecting the incident surface 150a and the rear surface 150b. The housing 150 has a substantially rectangular prism (box) shape, and has a substantially quadrilateral shape when viewed from the side on which radiation is incident.

In the present exemplary embodiment, the housing 150 includes an upper cover 151 (front surface member) and a lower cover 152 (rear surface member). Both the upper cover 151 and the lower cover 152 are in a tray shape having a bottom plate and a side wall. The lower cover 152 closes the opening of the upper cover 151, whereby the housing 150 in a box shape is configured, and a space for accommodating the internal component (internal space) is configured.

The bottom plate of the upper cover 151 corresponds to the incident surface 150a on which the radiation 211 is incident. Thus, it is desirable that a part of the upper cover 151 corresponding to the incident surface 150a be made of a material with low radiation absorption, for example, plastic, carbon, or carbon fiber reinforced plastic (CFRP).

The other parts (such as the side wall) of the upper cover 151 and the lower cover 152 may be made of the same material as the incident surface 150a, or may be made of a material having high rigidity, such as metal, alloy, and ceramic.

The sensor panel 101 is a radiation detection panel (radiation detection sensor, radiation detector) that converts radiation into an electrical signal. The sensor panel 101 has a substantially quadrilateral shape when viewed from the side on which radiation is incident. The sensor panel 101 includes a plurality of pixels, and each of the pixels generates an electric signal in accordance with the amount of incident radiation. The sensor panel 101 in the present exemplary embodiment is, for example, an indirect-type radiation detector including a phosphor that converts radiation into light, and has the following configuration. Each of the pixels of the sensor panel 101 has a plurality of layers. In the plurality of layers, a switching element, such as a thin film transistor (TFT), a photoelectric conversion unit made of amorphous silicon (a-Si), low temperature polysilicon (LTPS), an oxide semiconductor (IGZO), or the like, and a scintillator layer are layered on an insulating substrate, such as a glass substrate. The scintillator layer converts radiation into visible light, and the photoelectric conversion unit converts the visible light into an electric charge. The scintillator layer is made of cesium iodide (CsI), terbium doped gadolinium oxysulfide (GOS) (Gd2O2S: Tb), or the like. In particular, in a case of using CsI, thallium (Tl) or sodium (Na) is used as an activator. The scintillator layer is covered with a protective film made of, for example, polyparaxylylene (parylene), hot-melt resin, or a layered sheet of hot-melt resin and aluminum.

As the sensor panel 101, a direct-type radiation detector that converts radiation directly into an electrical signal may be used. In this case, each of the pixels of the sensor panel 101 includes a conversion unit that directly converts radiation into a charge, instead of including a scintillator layer. The conversion unit that directly converts radiation into a charge may be made of amorphous selenium (a-Se), cadmium telluride (CdTe), cadmium zinc telluride (CdZnTe), or the like.

The mounting board 190 is a circuit board on which an electrical component (not illustrated) is mounted. The electrical component includes at least one of an integrated circuit (for example, a drive circuit) that controls operation of the sensor panel 101 and an integrated circuit (for example, an amplifier integrated circuit (IC) or a reading circuit) that processes a signal from the sensor panel 101. While the mounting board 190 is represented by one board in FIG. 1, the mounting board 190 may include a plurality of boards. In this case, the plurality of boards may be disposed on the same plane or may be disposed to overlap one another in a height direction (z direction).

The sensor panel 101 and the mounting board 190 are connected with the cables 180 to exchange a signal. The cables 180 are, for example, a flexible printed circuit (FPC) (flexible printed circuit board, flexible board), and an electrical component, such as an integrated circuit 181 (IC chip), is disposed on the cables 180.

The base 110, the base 130, and the support columns 111 are support structures for stably supporting the sensor panel 101 and the mounting board 190 in the internal space of the housing 150. The base 130 is attached to the lower cover 152, and the base 110 is attached to the base 130 via the support columns 111.

The base 110 is a member that supports the sensor panel 101 and various boards. The base 110 has a substantially quadrilateral shape when viewed from the side on which radiation is incident. The sensor panel 101 is attached to the front surface (first surface) of the base 110 by a bonding member, such as an adhesive tape or an adhesive.

As a material of the base 110, a material having high rigidity and flatness is desirable, and for example, alloy, metal, resin, ceramic, or the like may be used. This makes it possible to stably support the sensor panel 101.

As a material of the base 130, metal or alloy having high thermal conductivity, resin containing filler, or the like may be used. With this configuration, heat generated by the mounting board 190 to the lower cover 152 is released.

As a material of the support columns 111, metal, alloy, a combination of materials, such as ceramic, or resin may be used. It is desirable to use a material having low thermal conductivity as the material of the support columns 111. While, in FIG. 1, the support columns 111 are illustrated in a cylindrical shape, the shape may be different as long as the support columns 111 connect the base 110 and the base 130.

The heat insulation members 160 and 161 are members for partitioning (separating) the internal space of the housing 150 into a space on a side with the incident surface 150a and a space on a side with the rear surface 150b. The heat insulation members 160 and 161 prevent air warmed by the mounting board 190 from flowing into a space where the sensor panel 101 is present (thermal convection), and reduce a temperature rise of the sensor panel 101. The heat insulation structure of the heat insulation members 160 and 161 may have some gaps or the like within a range in which a temperature rise of the sensor panel 101 is sufficiently reduced. However, it is desirable to minimize the gaps. The heat insulation members 160 and 161 will be described in detail below.

### [Heat Insulation Member]

The heat insulation structure in the present exemplary embodiment is divided into the heat insulation member 161 (first heat insulation member) and the heat insulation member 160 (second heat insulation member) which are configured as separate bodies, and has a structure in which the heat insulation members 160 and 161 hold the cables 180 therebetween when the upper cover 151 and the lower cover 152 are assembled.

The heat insulation member 161 is attached to the rear surface (second surface) of the base 110 in such a manner that the heat insulation member 161 is in a position between the sensor panel 101 and the mounting board 190. The heat insulation member 161 may be bonded to the base 110 via an adhesive layer (not illustrated), such as a double-sided tape or an adhesive, or may be physically fixed by a fastening member, such as a screw.

The heat insulation member 160 is attached to the inside surface of the side wall 150c of the housing 150. The heat insulation member 160 may be bonded to the housing 150 via an adhesive layer (not illustrated), such as a double-sided tape or an adhesive, or may be physically fixed by a fastening member, such as a screw.

In FIG. 1, the heat insulation members 160 and 161 have substantially the same thicknesses (length extending downward (z direction) on the drawing of FIG. 1). However, the heat insulation member 160 may be thicker than the heat insulation member 161, and the heat insulation member 161 may be thicker than the heat insulation member 160. The heat insulation members 160 and 161 may be configured in such a manner that in one region along the side wall 150c, one of the heat insulation members 160 and 161 is thicker than the other, and in the other region, the other is thinner.

In FIG. 1, the heat insulation members 160 and 161 hold the cables 180 therebetween in substantially the entire region in the thickness direction (z direction), but may hold the cables 180 therebetween in a partial region in the thickness direction.

The heat insulation members 160 and 161 desirably have a property of being resistant to heat transmission, and the heat conductivity is desirably about 0.01 to 0.5 W/(m·K).

Each of the heat insulation members 160 and 161 is, for example, a solid resin or a layered body, a filling material structure in which a liquid resin is cured, or a foamable resin having a high thermal insulation effect. As a material of each of the heat insulation members 160 and 161, a resin material, such as phenol resin, epoxy resin, silicon resin, acrylic resin, polyetheretherketone (PEEK) resin, polyethylene terephthalate (PET), vinyl chloride, polycarbonate, fluororesin, urethane resin, or rubber, may be used. The same material may be used for the heat insulation members 160 and 161, or different materials may be used for the heat insulation members 160 and 161.

As illustrated in FIG. 2A, when viewed from the side on which radiation is incident, the base 110 is larger than the sensor panel 101, and the heat insulation member 161 is larger than the base 110. That is, when viewed from the side on which radiation is incident, the heat insulation member 161 has a dimension (positional relationship) extending beyond the edge of the base 110. The heat insulation member 161 wider than the base 110 leads to lower likelihood of occurrence of a situation in which the cables 180 comes into contact with the edge of the base 110 and is damaged.

As illustrated in FIG. 2B, the heat insulation members 160 and 161 hold the cables 180 therebetween in close contact with each other when viewed in the cross-section taken along the line A-A. That is, in a region where no cable 180 is present between the heat insulation members 160 and 161, the heat insulation members 160 and 161 are in a relationship of direct contact with each other. In this way, the heat insulation members 160 and 161 are disposed without a gap in a certain cross section, and thus the internal space of the housing 150 is partitioned into the space on the side with the incident surface 150a and the space on the side with the rear surface 150b. In other words, the internal space of the housing 150 includes the space on the side with the incident surface 150a and the space on the side with the rear surface 150b on both sides of positions, which are interposed between the spaces, where the heat insulation members 160 and 161 hold the cables 180 therebetween.

As illustrated in FIG. 2B, the heat insulation member 161 has a hole (opening) for each support column 111 to pass through. In a case of the heat insulation member 161 made of a material having elasticity, the hole for the support column 111 to pass through is desirably smaller than the support column 111. With the support column 111 inserted into the hole which fits to the shape of the support column 111, there is no gap, whereby heat insulation is achieved. A double-sided tape or an adhesive layer may be disposed on a surface of the heat insulation member 161 which is in contact with the base 110. With such a configuration, misalignment of the heat insulation member 161 less likely occurs when the heat insulation member 161 is disposed on the base 110. In addition, the hole of the heat insulation member 161 and the support column 111 are used as positioning indexes, and thus the heat insulation member 161 is accurately disposed in a planar direction (a direction orthogonal to the radiation incident direction, that is, an x direction).

While the heat insulation member 160 is illustrated as a single member in FIG. 2B, a different configuration may be employed. For example, the heat insulation member 160 may be configured with a total of four members which are disposed on four sides each corresponding to a different side of the inner wall when viewed from the side on which radiation is incident. The heat insulation member 160 may be configured with a different number of a plurality of members.

In order to increase the heat insulation effect, it is desirable to appropriately bring the heat insulation members 160 and 161 into close contact with each other. Thus, in a configuration in which a foam member is used for the heat insulation members 160 and 161, it is desirable that the heat insulation members 160 and 161 are crushed (compressed) by the contact. For example, the volume after the contact is desirably smaller than the volume before the contact by 5% or more. In order to improve the tightness of the close contact, the contact surfaces of the heat insulation members 160 and 161 may be subjected to unevenness reduction processing or coating processing.

### [Ease of Assembly]

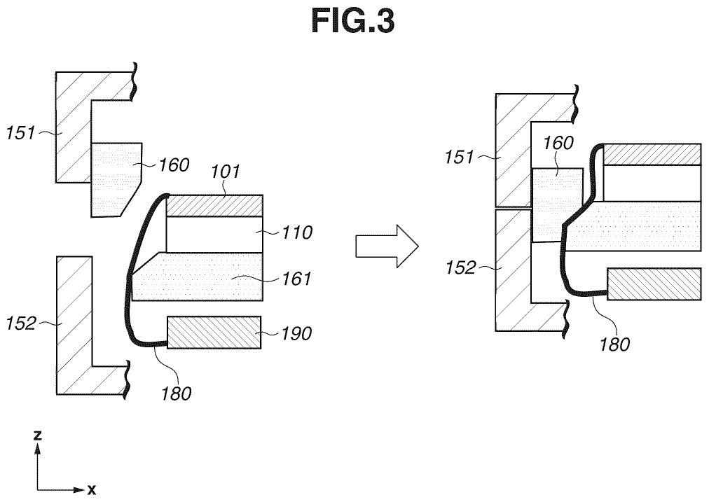

FIG. 3 is a schematic diagram illustrating the housing 150 during assembly. As described above, the heat insulation member 161 is disposed on the base 110, whereas the heat insulation member 160 is disposed on the housing 150. Thus, as illustrated on the left of FIG. 3, before the housing 150 is assembled, the heat insulation members 160 and 161 are not in close contact with each other, and the internal space of the housing 150 is not partitioned. Then, as illustrated in the right of FIG. 3, when the upper cover 151 and the lower cover 152 are fastened to each other, and the housing 150 is assembled, the heat insulation members 160 and 161 are brought into close contact with each other. Therefore, the internal space of the housing 150 is partitioned without an operation of passing each cable 180 through a hole in a heat insulation member.

In FIG. 3, to control deformation of the cables 180 during the assembly, each of the heat insulation members 160 and 161 has a tapered shape (rounded chamfer). With such a structure, the cables 180 are naturally deformed. Further, the heat insulation members 160 and 161 are prevented from being deformed in an unintended direction due to friction between the heat insulation members 160 and 161. The lower surface of the heat insulation member 160 is prevented from riding on the upper surface of the heat insulation member 161. Application of unintended stress to the cables 180 and a joint part between the cables 180 and the sensor panel 101 is prevented. That is, ease of assembly of the housing 150 is improved. From the viewpoint of ease of assembly, it is desirable that friction between the contact surfaces of the heat insulation members 160 and 161 be reduced by processing the contact surfaces.

In FIG. 3, the heat insulation member 160 is attached to the inner wall of the upper cover 151. This is because this configuration is desirable in terms of ease of assembly in a configuration in which the base 130, the support columns 111, and the base 110 are attached to or above the lower cover 152. If a method of attaching the base 110 is devised, and the degree of ease of assembly is acceptable, the heat insulation member 160 may be attached to or above the lower cover 152. Alternatively, without attaching the heat insulation member 160 to the housing 150, bringing the heat insulation member 160 into contact with the heat insulation member 161 and fastening the upper cover 151 and the lower cover 152 may be performed separately.

### [Effects]

As described above, the internal space of the housing 150 is partitioned into the space on the side with the incident surface 150a and the space on the side with the rear surface 150b by the heat insulation member 160 and the heat insulation member 161 holding the cables 180 therebetween. Thus, thermal convection is reduced, and a temperature rise of the sensor panel 101 is reduced.

A second exemplary embodiment will be described. In the first exemplary embodiment, simple structures of the heat insulation members 160 and 161 have been described. In the present exemplary embodiment, complex structures will be described as structures of the heat insulation members 160 and 161. The configuration of the present exemplary embodiment is substantially the same as that of the first exemplary embodiment except for a configuration that is a feature of the present exemplary embodiment. Thus, the same reference numeral is used for substantially the same component, and the redundant detailed description will be omitted.

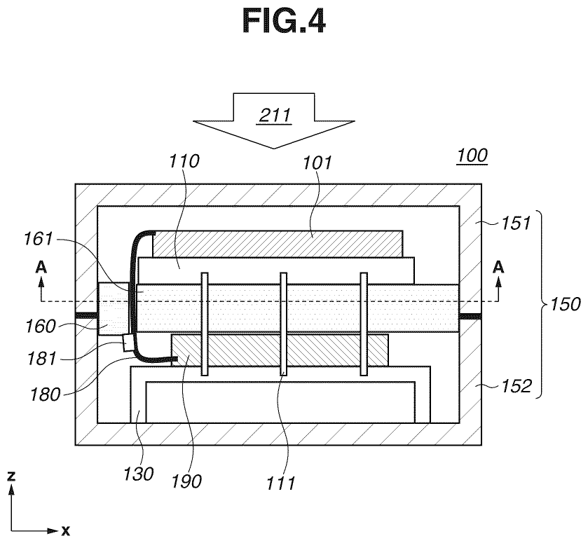

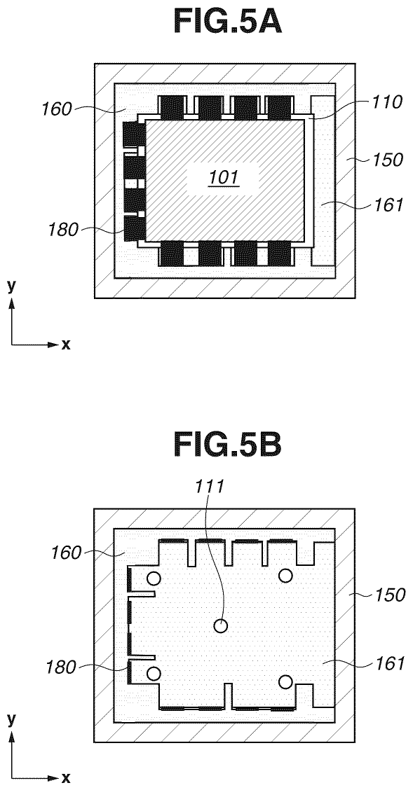

FIG. 4 is a cross-sectional diagram illustrating another example of an internal configuration of a radiation imaging apparatus 100 viewed from a side. FIG. 5A is a diagram illustrating the radiation imaging apparatus 100 viewed from a side on which radiation is incident, with an incident surface omitted. FIG. 5B is a diagram illustrating a cross section of the radiation imaging apparatus 100 taken along a line A-A illustrated in FIG. 4.

In FIG. 4, cables 180 are connected to the left side of a sensor panel 101, and the cables 180 is not connected to the right side of the sensor panel 101. Because the number of cables 180 connected to the sensor panel 101 depends on the number of wires in the sensor panel 101, such a configuration is also applicable. In the region where the cables 180 are absent, the internal space of a housing 150 is partitioned without bringing a heat insulation member 160 and a heat insulation member 161 into close contact with each other. Thus, in FIG. 4, the heat insulation member 161 is extended rightward and is brought into contact with a side wall 150c, whereby the internal space of the housing 150 is partitioned. As illustrated in FIG. 5A, on the right side of the four sides of the perimeter of the sensor panel 101, the heat insulation member 161 is in contact with at least a partial region (predetermined region) of the side wall 150c (either or both an upper cover 151 or/and a lower cover 152) facing the right side. The heat insulation member 160 is absent in this region. While the heat insulation member 160 is absent on one side alone in FIG. 5A, the heat insulation member 160 may be absent on a plurality of sides.

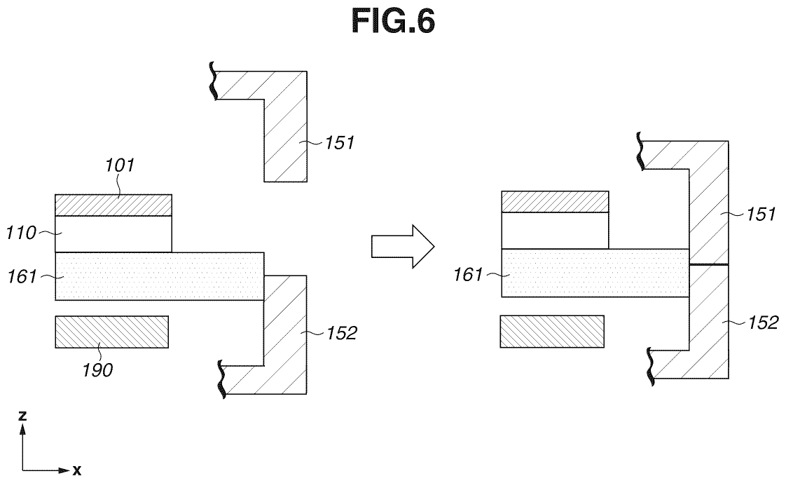

FIG. 6 is a schematic diagram illustrating assembly of the housing 150. As illustrated in the left of FIG. 6, the heat insulation member 161 is in contact with the lower cover 152. Then, as illustrated in the right of FIG. 6, when the upper cover 151 and the lower cover 152 are fastened to each other and the housing 150 is assembled, the upper cover 151 comes into contact with the heat insulation member 161. Thus, the internal space of the housing 150 is partitioned without an operation of passing each cable 180 through a hole in the heat insulation member 161.

As illustrated in FIG. 5B, the heat insulation members 160 and 161 each have a complex shape. Specifically, the heat insulation member 161 has protrusions (projection parts) each of which comes into contact with a different one of a plurality of cables 180 on the upper side of the four sides of the perimeter of the sensor panel 101.

Protrusions of the heat insulation member 160 are disposed to fit in recesses between the protrusions of the heat insulation member 161. While the structure is adopted on one side in FIG. 5B, the structure may be adopted on a plurality of the sides.

On the lower side of the four sides of the perimeter of the sensor panel 101, the heat insulation member 161 has protrusions (projection parts) each of which is for every two different cables among the plurality of cables 180. Protrusions of the heat insulation member 160 are disposed to fit in recesses between the protrusions of the heat insulation member 161. While the structure is adopted on one side in FIG. 5B, the structure may be adopted on a plurality of the sides.

On the left side of the four sides of the perimeter of the sensor panel 101, the heat insulation member 161 has protrusions (projection parts). Among those, one type of the protrusions comes into contact with a different one of the plurality of cables 180, and the other type of the protrusions is disposed for every two different cables among the plurality of cables 180. Protrusions of the heat insulation member 160 are disposed to fit in recesses between the protrusions of the heat insulation member 161. While the structure is adopted on one side in FIG. 5B, the structure may be adopted on a plurality of the sides.

### [Effects]

As described above, devising the shapes and the placement of the heat insulation members 160 and 161 is expected to result in a further heat insulation effect.

### Other Embodiments

The present disclosure is not limited to the above-described exemplary embodiments, and various modifications (including organic combinations of the exemplary embodiments) may be made based on the spirit of the present disclosure, and these modifications are not excluded from the scope of the present disclosure. That is, all configurations obtained by combining the above-described exemplary embodiments and modifications of the above-described exemplary embodiments are also included in the present disclosure.

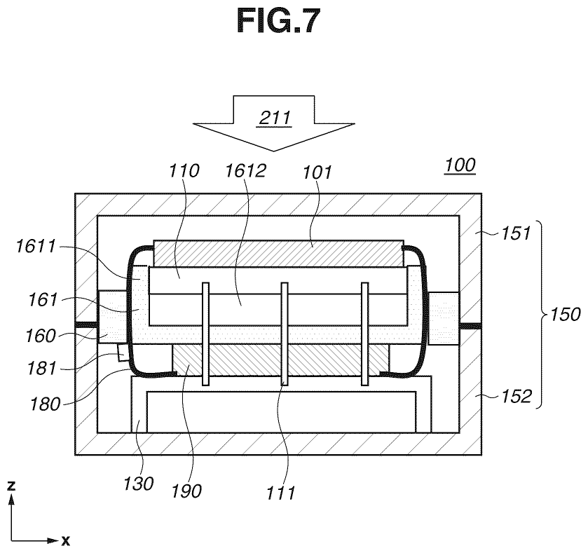

In the first and second exemplary embodiments, the heat insulation member 161 that has a simple cross section in the thickness direction (z direction) and is configured separately from the base 110 has been described. However, the structure of the heat insulation member 161 is not limited to this. FIG. 7 is a cross-sectional diagram illustrating yet another example of an internal configuration of a radiation imaging apparatus 100 viewed from a side. As illustrated in FIG. 7, the heat insulation member 161 and a base 110 are separated from each other with a space 1612. With such a configuration, a further heat insulation effect may be expected.

Alternatively, an additional member (for example, a member having a high X-ray shielding property, such as lead (Pb), to improve the X-ray shielding property) may be disposed instead of the space 1612. Further, a side wall part 1611 in contact with a side surface of the base 110 may be disposed. With such a configuration, any cable 180 is prevented from being damaged due to contact between the cable 180 and an edge of the base 110.

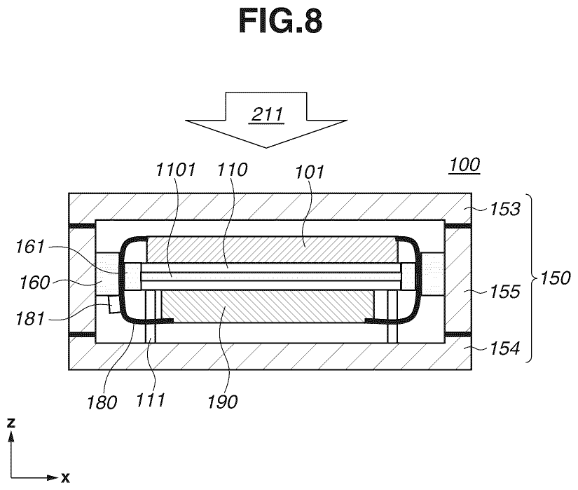

In the first and second exemplary embodiments, the heat insulation member 161 is disposed on the second surface of the base 110. However, the structure of the heat insulation member 161 is not limited to this. FIG. 8 is a cross-sectional diagram illustrating yet another example of an internal configuration of a radiation imaging apparatus 100 viewed from a side. As illustrated in FIG. 8, a configuration in which a heat insulation member 161 is disposed between a side surface of a base 110 and a cable 180 may be employed. This configuration contributes to a reduction in the thickness of the radiation imaging apparatus 100. In a case of this configuration, the base 110 desirably has a composite layered structure including a heat insulating layer. In the case of using the composite layered structure, a mounting board 190 may be directly attached to the base 110. A housing 150 may be configured with three members of an upper cover 153, a lower cover 154, and a side wall frame 155 (side wall member), instead of two members of the upper cover 151 and the lower cover 152. With this configuration, when a heat insulation member 160 and the heat insulation member 161 are brought into close contact with each other, an assembly method with a higher degree of freedom may be used. The base 110 may be attached to the lower cover 154 with support columns 111.

In the first exemplary embodiment, the mounting board 190 is disposed between the base 110 and the base 130. Alternatively, the mounting board 190 may be disposed between the base 130 and the lower cover 154 and attached to the lower cover 154.

### [Appendix]

The disclosure of the present exemplary embodiments includes the following configurations.

### [Appendix 1]

A radiation imaging apparatus comprising:
a sensor panel configured to detect radiation;
a cable configured to be connected to the sensor panel;
a board configured to be connected to the sensor panel via the cable;
a plurality of heat insulation members which are separate members including a first heat insulation member and a second heat insulation member; and
a housing configured to house the sensor panel, the cable, the board, and the plurality of heat insulation members, the housing including an incident surface through which radiation is incident on the sensor panel, a rear surface on a side opposite to the incident surface, and a side wall connecting the incident surface and the rear surface;
wherein the first heat insulation member is disposed between the sensor panel and the board,
wherein the second heat insulation member is disposed between the cable and the side wall, and
wherein an internal space of the housing includes a space on a side with the incident surface and a space on a side with the rear surface, between which is a position where the first heat insulation member and the second heat insulation member hold the cable therebetween.

### [Appendix 2]

The radiation imaging apparatus according to Appendix 1, further comprising: a support base configured to support the sensor panel on a first surface;
wherein the first heat insulation member is disposed to a second surface of the support base, the second surface being opposite the first surface.

### [Appendix 3]

The radiation imaging apparatus according to Appendix 2,
wherein when viewed from a side on which radiation is incident, a positional relationship in which a part of the first heat insulation member protrudes from a perimeter of the support base is satisfied.

### [Appendix 4]

The radiation imaging apparatus according to Appendix 2 or 3,
wherein the first heat insulation member has a part which is in contact with a side surface of the support base.

### [Appendix 5]

The radiation imaging apparatus according to any one of Appendices 2 to 4,
wherein the support base is provided with a support column for attaching the board, and
wherein the first heat insulation member has an opening through which the support column is passed.

### [Appendix 6]

The radiation imaging apparatus according to any one of Appendices 2 to 5,
wherein the first heat insulation member is disposed at a position spaced apart from the second surface of the support base with a gap between the first heat insulation member and the support base.

### [Appendix 7]

The radiation imaging apparatus according to any one of Appendices 2 to 6, further comprising:
a support base configured to support the sensor panel on the first surface;
wherein the first heat insulation member is disposed on a side surface of the support base.

### [Appendix 8]

The radiation imaging apparatus according to any one of Appendices 2 to 7,
wherein the support base has a composite layered structure including a heat insulation layer.

### [Appendix 9]

The radiation imaging apparatus according to any one of Appendices 1 to 8,
wherein a material of the first heat insulation member and the second heat insulation member is resin or foamable resin.

### [Appendix 10]

The radiation imaging apparatus according to any one of Appendices 1 to 9,
wherein the first heat insulation member and the second heat insulation member are compressed by holding the cable therebetween.

### [Appendix 11]

The radiation imaging apparatus according to any one of Appendices 1 to 10,
wherein the cable is a flexible board including an integrated circuit (IC) chip, and
wherein, in a state where the first heat insulation member and the second heat insulation member hold the cable therebetween, the IC chip is in the space on the side with the rear surface.

### [Appendix 12]

The radiation imaging apparatus according to any one of Appendices 1 to 11, further comprising:
another cable configured to be connected to the sensor panel,
wherein in a region between the cable and the another cable, a part of the first heat insulation member is in contact with a part of the second heat insulation member.

### [Appendix 13]

The radiation imaging apparatus according to any one of Appendices 1 to 12, further comprising:
a plurality of cables configured to include the cable;
wherein at least a part of the second heat insulation member has a protrusion disposed between adjacent cables of the plurality of cables, and the protrusion is fitted into a recess in the first heat insulation member.

### [Appendix 14]

The radiation imaging apparatus according to any one of Appendices 1 to 13,
wherein the housing includes a front member having the incident surface and a rear member having the rear surface, and
wherein the second heat insulation member is in contact with both the front member and the rear member.

### [Appendix 15]

The radiation imaging apparatus according to any one of Appendices 1 to 14,
wherein the housing includes a front member having the incident surface and a rear member having the rear surface, and
wherein the second heat insulation member is attached to one of the front member and the rear member.

### [Appendix 16]

The radiation imaging apparatus according to any one of Appendices 1 to 15,
wherein the housing includes a front member having the incident surface, a rear member having the rear surface, and a side wall member connecting the front member and the rear member, and
wherein the second heat insulation member is attached to the side wall member.

### [Appendix 17]

The radiation imaging apparatus according to any one of Appendices 1 to 16, further comprising:
a plurality of cables configured to include the cable;
wherein when viewed from the side on which radiation is incident, and a perimeter of the sensor panel is seen as a quadrilateral shape, the sensor panel has a first side to which any of the plurality of cables is connected and a second side to which no cable of the plurality of cables is connected.

### [Appendix 18]

The radiation imaging apparatus according to any one of Appendices 1 to 17, wherein the second heat insulation member is disposed on a side with the first side, and the second heat insulation member is absent on a side with the second side.

### [Appendix 19]

The radiation imaging apparatus according to Appendix 18, wherein the first heat insulation member is in contact with a predetermined region of the side wall facing the second side.

### [Appendix 20]

A radiation imaging system comprising:
a radiation imaging apparatus according to any one of Appendices 1 to 19, and
a signal processing unit configured to process a signal obtained by the radiation imaging apparatus.

### [Appendix 21]

A manufacturing method of a radiation imaging apparatus including a sensor panel configured to detect radiation, a cable configured to be connected to the sensor panel, a board configured to be connected to the sensor panel via the cable, a plurality of heat insulation members which are separate members including a first heat insulation member and a second heat insulation member, and a housing configured to house the sensor panel, the cable, the board, and the plurality of heat insulation members, the housing including an incident surface through which radiation is incident on the sensor panel, a rear surface on a side opposite to the incident surface, and a side wall connecting the incident surface and the rear surface, the manufacturing method comprising:
disposing the first heat insulation member between the sensor panel and the board,
disposing the second heat insulation member between the cable and the side wall, and
holding the cable between the first heat insulation member and the second heat insulation member in such a manner that an internal space of the housing includes a space on a side with the incident surface and a space on a side with the rear surface.

While the present disclosure includes exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2024-063718, filed Apr. 11, 2024, which is hereby incorporated by reference herein in its entirety.

What is claimed is:

1. A radiation imaging apparatus comprising:
a sensor panel configured to detect radiation;
a cable configured to be connected to the sensor panel;
a board configured to be connected to the sensor panel via the cable;
a plurality of heat insulation members which are separate members including a first heat insulation member and a second heat insulation member; and
a housing configured to house the sensor panel, the cable, the board, and the plurality of heat insulation members, the housing including an incident surface through which radiation is incident on the sensor panel, a rear surface on a side opposite to the incident surface, and a side wall connecting the incident surface and the rear surface;
wherein the first heat insulation member is disposed between the sensor panel and the board,
wherein the second heat insulation member is disposed between the cable and the side wall, and
wherein an internal space of the housing includes a space on a side with the incident surface and a space on a side with the rear surface, between which is a position where the first heat insulation member and the second heat insulation member hold the cable therebetween.

2. The radiation imaging apparatus according to claim 1, further comprising:
a support base configured to support the sensor panel on a first surface,
wherein the first heat insulation member is disposed to a second surface of the support base, the second surface being opposite the first surface.

3. The radiation imaging apparatus according to claim 2,
wherein when viewed from a side on which radiation is incident, a positional relationship in which a part of the first heat insulation member protrudes from a perimeter of the support base is satisfied.

4. The radiation imaging apparatus according to claim 2,
wherein the first heat insulation member has a part which is in contact with a side surface of the support base.

5. The radiation imaging apparatus according to claim 2,
wherein the support base is provided with a support column for attaching the board, and
wherein the first heat insulation member has an opening through which the support column is passed.

6. The radiation imaging apparatus according to claim 2,
wherein the first heat insulation member is disposed at a position spaced apart from the second surface of the support base with a gap between the first heat insulation member and the support base.

7. The radiation imaging apparatus according to claim 2,
wherein the support base has a composite layered structure including a heat insulation layer.

8. The radiation imaging apparatus according to claim 1, further comprising:
a support base configured to support the sensor panel on a first surface,
wherein the first heat insulation member is disposed on a side surface of the support base.

9. The radiation imaging apparatus according to claim 1,
wherein a material of the first heat insulation member and the second heat insulation member is resin or foamable resin.

10. The radiation imaging apparatus according to claim 1,
wherein the first heat insulation member and the second heat insulation member are compressed by holding the cable therebetween.

11. The radiation imaging apparatus according to claim 1,
wherein the cable is a flexible board including an integrated circuit (IC) chip, and
wherein, in a state where the first heat insulation member and the second heat insulation member hold the cable therebetween, the IC chip is in the space on the side with the rear surface.

12. The radiation imaging apparatus according to claim 1, further comprising:
another cable configured to be connected to the sensor panel,
wherein in a region between the cable and the another cable, a part of the first heat insulation member is in to contact with a part of the second heat insulation member.

13. The radiation imaging apparatus according to claim 1, further comprising:
a plurality of cables configured to include the cable,
wherein at least a part of the second heat insulation member has a protrusion disposed between adjacent cables of the plurality of cables, and the protrusion is fitted into a recess in the first heat insulation member.

14. The radiation imaging apparatus according to claim 1,
wherein the housing includes a front member having the incident surface and a rear member having the rear surface, and
wherein the second heat insulation member is in contact with both the front member and the rear member.

15. The radiation imaging apparatus according to claim 1,
wherein the housing includes a front member having the incident surface and a rear member having the rear surface, and
wherein the second heat insulation member is attached to one of the front member and the rear member.

16. The radiation imaging apparatus according to claim 1,
wherein the housing includes a front member having the incident surface, a rear member having the rear surface, and a side wall member connecting the front member and the rear member, and
wherein the second heat insulation member is attached to the side wall member.

17. The radiation imaging apparatus according to claim 1, further comprising:
a plurality of cables configured to include the cable,
wherein when viewed from the side on which radiation is incident, and a perimeter of the sensor panel is seen as a quadrilateral shape, the sensor panel has a first side to which any of the plurality of cables is connected and a second side to which no cable of the plurality of cables is connected.

18. The radiation imaging apparatus according to claim 17, wherein the second heat insulation member is disposed on a side with the first side, and the second heat insulation member is absent on a side with the second side.

19. The radiation imaging apparatus according to claim 18, wherein the first heat insulation member is in contact with a predetermined region of the side wall facing the second side.

20. A manufacturing method of a radiation imaging apparatus including a sensor panel configured to detect radiation, a cable configured to be connected to the sensor panel, a board configured to be connected to the sensor panel via the cable, a plurality of heat insulation members which are separate members including a first heat insulation member and a second heat insulation member, and a housing configured to house the sensor panel, the cable, the board, and the plurality of heat insulation members, the housing including an incident surface through which radiation is incident on the sensor panel, a rear surface on a side opposite to the incident surface, and a side wall connecting the incident surface and the rear surface, the manufacturing method comprising:
disposing the first heat insulation member between the sensor panel and the board;
disposing the second heat insulation member between the cable and the side wall; and
holding the cable between the first heat insulation member and the second heat insulation member in such a manner that an internal space of the housing includes a space on a side with the incident surface and a space on a side with the rear surface.
